(19) United States
(12) Patent Application Publication
(10) Pub. No.: US 2011/0060120 A1
(43) Pub. Date: Mar. 10, 2011

(54) IMMUNOGENIC TREATMENT OF CANCER BY PEPTIDES INDUCING THE PLASMA MEMBRANE EXPOSURE OF ERP57

(76) Inventor: Michel Sarkis OBEID, Paris (FR)

(21) Appl. No.: 12/882,183

(22) Filed: Sep. 14, 2010

Related U.S. Application Data
(63) Continuation-in-part of application No. 11/774,585, filed on Jul. 7, 2007, now abandoned.
Application No. 11/774,585 is a continuation-in-part of application No. 11/845,060, filed on Aug. 25, 2007, now abandoned.
Application No. 11/845,060 is a continuation-in-part of application No. 11/845,061, filed on Aug. 25, 2007.
Application No. 11/845,061 is a continuation-in-part of application No. 11/845,062, filed on Aug. 25, 2007, now abandoned.
Application No. 11/845,062 is a continuation-in-part of application No. 11/845,063, filed on Aug. 25, 2007, now abandoned.
Application No. 11/845,063 is a continuation-in-part of application No. 11/845,064, filed on Aug. 25, 2007, now abandoned.
Application No. 11/845,064 is a continuation-in-part of application No. 11/845,065, filed on Aug. 25, 2007.
Application No. 11/845,065 is a continuation-in-part of application No. 11/845,067, filed on Aug. 25, 2007, now abandoned.
Application No. 11/845,067 is a continuation-in-part of application No. 11/845,068, filed on Aug. 26, 2007, now abandoned.
Application No. 11/845,068 is a continuation-in-part of application No. 11/845,069, filed on Aug. 26, 2007.

(30) Foreign Application Priority Data
Sep. 8, 2006 (EP) 06291427.0-2107
Aug. 31, 2007 (IB) PCT/IB2007/002502
Mar. 5, 2009 (CA) 2,665,771

(51) Int. Cl. C07K 2/00

(52) U.S. Cl. 530/300

(57) ABSTRACT

We have recently identified (a) ectocalreticulin as the main source of immunogenicity of cancer cell death induced by chemotherapy or radiotherapy, (b) ectoERP57 as critical protein for inducing cell surface exposure of calreticulin, and (c) that ectoERP57 and ectocalreticulin are cotranslocated together to the tumor cell surface by the mediator of the inhibition of PP1/GADD34 complex. Here, I show the design of a peptide that inhibits the interaction between PP1 and GADD34 complex. These inhibitor peptide (a) induce ectocalreticulin and ectoERP57 in a variety of tumor cell lines by the mediator of the inhibition of the interaction between PP1 and GADD34, (b) increase the phagocytosis of anticancer targeted proapoptotic peptide and chemotherapy-treated tumor cells by dendritic cells, and (c) improve highly the anticancer activity of proapoptotic peptides and chemotherapy by suppressing or reducing the tumor growth in several isogenic mouse models of colon, mammary, and fibrosarcoma tumors and by increasing the lifespan of transgenic adenocarcinoma mouse prostate mice. These results suggest that these targeted peptides combination approach could serve as a new powerful autonomous anticancer therapy.

### PRIORITY CLAIM

The present application claims the priority of the following patent applications, which are incorporated herein by this reference in their entirety:

The European patent application, Serial No. 06291427.0-2107, filed on Sep. 8, 2006, titled “Calreticulin for its Use as a Medication For The Treatment of a Disease Such as Cancer in a Mammal”.

The Canadian patent application, Serial No. 2,665,771, filed on Mar. 5, 2009, titled “Method and Kit for Effecting Screening and Immunogenic Treatment Using CRT and/or ERP57 Translocation”.

The PCT patent application, No. PCT/IB2007/002502, filed on Aug. 31, 2007.

The present application claims the priority of, and is a continuation-in-part application of the following patent applications, which are incorporated herein by this reference in their entirety:

The American patent application, Ser. No. 11/774,585, filed on Jul. 7, 2007, titled “Method, Apparatus, and Compound for Effecting Localized, Non-Systemic, Immunogenic Treatment of Cancer”.

U.S. patent application, Ser. No. 11/845,060, filed on Aug. 25, 2007, titled “Method for Effecting Localized, Non-Systemic and Systemic, Immunogenic Treatment of Cancer Using CRT Translocation”.

U.S. patent application, Ser. No. 11/845,061, filed on Aug. 25, 2007, titled “Method for Effecting Localized, Non-Systemic and Systemic, Immunogenic Treatment of Cancer Using ERP57 Translocation”.

U.S. patent application, Ser. No. 11/845,062, filed on Aug. 25, 2007, titled “A Pharmaceutical Compound for Effecting Localized, Non-Systemic and Systemic, Immunogenic Treatment of Cancer Using CRT or ERP57 Translocation”.

U.S. patent application, Ser. No. 11/845,063, filed on Aug. 25, 2007, titled “A Pharmaceutical Compound for Blocking the CRT or ERP57 Translocation”.

U.S. patent application, Ser. No. 11/845,064, filed on Aug. 25, 2007, titled “A Kit for Treating a Health Condition by Inducing Translocation of a Calreticulin Protein to a Cellular Membrane”.

U.S. patent application, Ser. No. 11/845,065, filed on Aug. 25, 2007, titled “A Kit for Treating a Health Condition by Inducing Translocation of an ERP57 Protein to a Cellular Membrane”.

U.S. patent application, Ser. No. 11/845,067, filed on Aug. 25, 2007, titled “Service for Effecting Localized, Non-Systemic and Systemic, Immunogenic Treatment of Cancer Using CRT Translocation”.

U.S. patent application, Ser. No. 11/845,068, filed on Aug. 25, 2007, titled “Service for Effecting Localized, Non-Systemic and Systemic, Immunogenic Treatment of Cancer Using ERP57 Translocation”.

U.S. patent application, Ser. No. 11/845,069, filed on Aug. 25, 2007, titled “Method for Screening Fertility and New Compounds or Molecules, Using CRT and/or ERP57 Translocation”.

### FIELD OF THE INVENTION

The present invention relates to a new class of PP1/GADD34 inhibitor that is able to inhibit the interaction between PP1 and GADD34 contrarily to the existing inhibitors that inhibit only the activity of the PP1/GADD34 complex and not the interaction between PP1 and GADD34. The invention also deals with a peptide that inhibits the interaction between PP1 and GADD34 and consequently induces ERP57 exposure at the plasma membrane (ectoERP57) for a novel use as a medication in the treatment of diseases such as cancer.

### BACKGROUND OF THE INVENTION

Cancer is a leading cause of death worldwide. Principal treatment methods are: surgery, radiotherapy, immunotherapy, hormonotherapy and chemotherapy. However, chemotherapy is the main treatment when the cancer becomes metastatic. Chemotherapy leads to systemic tumor cell death. Two type of cell death are known: the apoptosis and the necrosis.

It has long been hypothesized that apoptotic cell death would be poorly immunogenic (or even tolerogenic) whereas necrotic cell death would be truly immunogenic.

This difference was thought to result from the intrinsic capacity of cells dying from non-apoptotic cell death to stimulate the immune response, for example by stimulating local inflammatory responses (‘danger signals’) and/or by triggering the maturation of dendritic cells (DCs).

In contrast to necrosis (which is defined by brisk plasma membrane rupture), apoptosis is associated with a series of subtle alterations in the plasma membrane that render the dying cells palatable to phagocytic cells. Such “eat me” signals, which include the adsorption of soluble proteins from outside the cell (such as C1q and thrombospondin) and the translocation of molecules from inside the cell to the surface (such as phosphatidylserine, PS, and calreticulin, CRT), as well, as the suppression of “don't eat me” signals (such as CD47) elicit the recognition and removal of apoptotic cells by professional and non-professional phagocytes. Suboptimal clearance of apoptotic cells can trigger unwarranted immune reactions and lead to autoimmune disease.

Nonetheless, it seems that the dichotomy between immunogenic necrosis versus tolerogenic apoptosis is an oversimplification. Thus, unscheduled (necrotic) tumor cell death may induce local immunosuppression. Moreover, the capacity of apoptotic tumor cells to trigger the immune response was found to depend on the apoptosis inducer, leading to the identification of two morphologically undistinguishable subcategories of apoptosis, namely immunogenic versus non-immunogenic.

Most of standard chemotherapies induce a non-immunogenic apoptosis. Thus, even after an initially efficient chemotherapy, patients do not develop an efficient antitumorous immune response and then are overcome by chemotherapy-resistant tumorous variants. To improve anticancer chemotherapy, a promising way is brought by the immunogenic cancer-cell death. Indeed, induction of immunogenic cancer-cell death should be very interesting in that the immune system can contribute through a “bystander effect” to eradicate chemotherapy-resistant cancer cells and cancer stem cells.

The efficiency of a chemotherapy and the responsiveness is relating to drugs used and the molecules involved in the chemotherapy. The main drugs used in anti-tumorous chemotherapy can be divided in four groups: cytotoxic agents, hormones, immune response modulators and inhibitors of the kinase tyrosin activity. It has been shown for the first time that anthracyclins are capable of eliciting immunogenic apoptosis. Indeed, while most apoptosis inducers, including agents that target the endoplasmic reticulum (ER) (thapsigargin, tunicamycin, and brefeldin), mitochondria (arsenite, betulinic acid, C2 ceramide) or DNA (Hoechst 33343, camptothecin, etoposide, mitomycin C), failed to induce immunogenic apoptosis, anthracycline elicited immunogenic cell death. Despite a growing body of research, under which circumstances an immune response is triggered against dying tumor cells remains an open question.

It has been an ongoing conundrum which particular biochemical change would determine the distinction between immunogenic and non-immunogenic cell death.

The present invention is based on the observation that the protein named ERP57 is present on plasma membrane of cells that succumb to immunogenic cell death, yet lacks on the surface of cells that undergo non-immunogenic cell death.

ERP57 is a soluble luminal protein of the endoplasmic reticulum (ER) and a protein disulfide isomerase (PDI) ortholog that forms complexes with calreticulin and calnexin. ERP57 intervenes in the disulfide bond formation in glycosylated proteins.

ERP57 translocated to the plasma membrane is named “ectoERP57”. Calreticulin (CRT) translocated to the plasma membrane is named “ectoCRT”.

Actually, a particular alteration was identified in the plasma membrane of dying cells: the plasma membrane exposure of ERP57 (ectoERP57). This event occurs exclusively in immunogenic cancer cell death but not in non-immunogenic cancer cell death.

Hence, the present invention concerns a peptide that induces ectoERP57 for its use as a medication for the treatment of cancer in a mammal, said medication inducing an increased quantity of ectoERP57.

The present invention is based on the identification of ectoERP57 as a determining feature of anti-cancer immune responses and delineates a strategy of immunogenic chemotherapy.

Furthermore, it has been shown that the ectoERP57 present in an increased amount renders the dying cells palatable to phagocytic cells such as dendritic cells. These cells interact with the immune system and then induce an immune response that renders the ERP57 as an inducer of immunogenic apoptosis. The present invention also concerns ectoERP57 for its use as a medication for the treatment of a disease in a mammal, said medication inducing an immunogenic apoptosis.

Preferably, by this application as a medication, the disease treated is a cancer such as breast cancer, prostate cancer, melanoma, colon cancer, etc.

The present invention exposes ERP57 for its use as a medication for the treatment of cancer in a mammal, said medication improving the efficiency of chemotherapy in a mammal in need of such chemotherapy by inducing an increased location of ectoERP57 and/or induction of immunogenic apoptosis.

### DETAILED DESCRIPTION OF THE INVENTION

### Immunogenic Cell Death

The present invention demonstrates that ectoERP57 dictates the immunogenicity of cancer cell death. The exposure of ERP57 on the plasma membrane of tumor cells is perceptible by Dendritic Cells (DCs) as “eat signal” that promotes their phagocytosis by DCs. This phagocytosis will elicit an immune response against tumor cells.

The response developed by the immune system is herein called “anti-cancer immune response”.
The new treatment strategy is herein called “immunogenic treatment of cancer”, when it is directed against a cancer.

The kinetic of ectoERP57 and ectocalreticulin (CRT) exposure is identical. The exposure of ectoCRT was abrogated when the intracellular expression of ERP57 was abolished and vice versa. Additionally, ectoCRT was co-immunoprecipitated with ectoERP57 and vice versa. Hence, ectoCRT and ectoERP57 are translocated together in the same molecular complex.

Unlike ectoCRT, ectoERP57 does not possess any immunogenic property.

The intensity of the anti-cancer immune response is proportional to the quantity of ectoERP57. The more the ectoERP57 quantity, the more the anti-cancer immune response.

Abolishing the exposure of ectoCRT by depleting the intracellular expression of CRT abrogates the immunogenicity of tumor cells treated by inhibitor peptide of the PP1/GADD34 complex (as shown in FIG. 5A). This immunogenicity was restored after the addition of recombinant CRT on the cell surface of tumor cells (as shown in FIG. 5B). Like the depletion of CRT, the depletion of intracellular ERP57 abolished the immunogenicity of tumor cells treated by inhibitor peptide of the PP1/GADD34 complex (as shown in FIG. 5C). This immunogenicity was restored after the addition of recombinant CRT (as shown in FIG. 5C).

The immunogenicity and the immune response are mediated by specific cells: dendritic cells (DCs). It has been shown that tumor cells treated with the inhibitor peptide of the complex PP1/GADD34 acquired the property to be phagocytosed by DCs (as shown in FIGS. 6A and B), correlating with the rapid induction of ectoERP57 (as shown in FIG. 6A).

Accordingly, blocking the ectoCRT present on the surface of cancer cells treated with inhibitor peptide of the PP1/GADD34 complex by means of a specific antibody inhibited their phagocytosis by DC (as shown in FIG. 6B) similarly to the depletion of intracellular CRT expression (as shown in FIG. 6C).

### PP1 and GADD34 Interacting Inhibitor

It has been shown that anthracycline such as doxorubicin, idarubicin and mitoxantrone; and PP1/GADD34 inhibitors such as salubrinal, calyculin A and tautomycin induces ectoERP57 by inhibiting the activity of PP1/GADD34 complex.

It has also been shown that anthracycline such as doxorubicin, idarubicin and mitoxantrone; and PP1/GADD34 inhibitors such as salubrinal, calyculin A and tautomycin are only inhibitors of the activity of PP1/GADD34 and not of the interaction between PP1 and GADD34.

The inhibitors of the activity of PP1/GADD34 such as salubrinal, calyculin A and tautomycin does not possess any anticancer activity in contrast to the inhibitor of the interaction between PP1 and GADD34 (consisting of a new class of inhibitor developed in this invention) which posses significant anti-cancer activity and are more immunogenic than salubrinal, calyculin A and tautomycin.

Accordingly, the present invention concerns an inhibitor of the interaction between PP1 and GADD34, for its use as a medication for the treatment of cancer in a mammal, said inhibitor inducing an increased location of endogenous ERP57 at the cellular surface.

Furthermore, the present invention is directed to an inhibitor of the interaction between PP1 and GADD34, for its use as a medication for the treatment of a disease in a mammal, said inhibitor inducing an immunogenic apoptosis by increased ectoERP57 translocation (as shown in FIGS. 2, 3 and 4).

It has been observed that ectoERP57 exposure induced by an inhibitor of the interaction between PP1 and GADD34 improves the anti-tumor immune response.

This present invention also concerns an inhibitor of the interaction between PP1 and GADD34 for its use as a medication for the treatment of a disease in a mammal, said inhibitor improving the efficiency of chemotherapy in a mammal in need of such chemotherapy by inducing an increased location of ectoERP57 and/or an immunogenic apoptosis (as shown in FIGS. 3 and 4).

Moreover, an amount of such inhibitor of the interaction between PP1 and GADD34 described above can be used in a pharmaceutical composition promoting an increased translocation of the ERP57 protein from the cytoplasm to the cell membrane which thus induces an immune response during apoptosis in a mammal.

Said inhibitor-comprised pharmaceutical composition promoting an increased translocation of the ERP57 from the cytoplasm to the cell surface can also ameliorate chemotherapy response in a mammal.

The present invention also concerns a product containing a chemotherapeutic agent and inhibitor of the interaction between PP1 and GADD34 as a combination product for its use in the treatment of cancer.

The chemotherapeutic agent could be anthracycline and others well known in therapeutics.

This method shows increased efficiency of chemotherapy in a mammal in need of such chemotherapy.

Preferably, the mammal treated is a human.

There is a basal amount of ectoERP57 and the increased amount of ectoERP57 allows predicting an immunogenic apoptosis and/or a therapeutic efficiency of chemotherapy. By comparing before and after chemotherapy, the amount of ectoERP57 is generally largely increased and the increase will be a good predictive marker for immunogenic apoptosis, therapeutic efficiency of a chemotherapy.

### BRIEF DESCRIPTION OF THE DRAWINGS

FIG. 1 shows the design of the panel of chimeric peptides.

FIG. 1A shows the five peptide domains used in this study. ProstTarget, which can target the prostate through a vascular address; BiP, which can target the tumor antigen BiP/GRP78; PTD, a protein transduction domain-5; PP1, an inhibitor peptide of the interaction between PP1 and GADD34;and KLAK, an antimicrobial peptide that induces apoptosis in several cell lines.

FIG. 1B shows the family of targeted proapoptotic peptides that contains three members. The first member is the chimeric peptide ProsTtarget/KLAK, which is composed of the ProstTarget peptide coupled to KLAK peptide separated by diglyicine spacer. The second member is the chimeric peptide BiP/KLAK, which is composed of BiP peptide coupled to KLAK peptide, separated by diglyicine spacer. The third member is the chimeric peptide DP1 (PTD/KLAK), which is composed of PTD peptide coupled KLAK, separated by diglyicine.

FIG. 1C shows the family of targeted ectoERP57-inducer peptides contains three members. The first member is the chimeric peptide ProstTarget/PP1, which is composed of ProstTarget peptide coupled to PP1 inhibitor peptide, separated by diglyicine spacer. The second member is the chimeric peptide composed of BiP peptide coupled to PP1 inhibitor peptide BiP/PP1, separated by diglyicine spacer. The third member is the chimeric peptide PTD/PP1, which is composed of PTD peptide coupled to PP1 inhibitor peptide, separated by diglyicine spacer.

FIG. 2. The chimeric inhibitor peptides inhibit the interaction between PP1 and GADD34 and induce ectocalreticulin and ectoERP57.

FIG. 2A. Interaction between PP1 and GADD34 were inhibited by chimeric inhibitor peptides. Untreated CT26, MCA205, and EF43.fgf4 cells or those treated 4 h with BiP/PP1 or PTD/PP1 were lysed after the treatment and immunoprecipitated with the anti-PP1 antibody. The levels of PP1 and GADD34 were analyzed by Western blot.

FIG. 2B. Arbitrary densitometry analysis for the immunoprecipitated PP1/GADD34 ratio before and after PTD/PP1 or BiP/PP1 treatment. Results are triplicates from one experiment (mean±SD) and are representative of three independent experiments. *, P<0.001 (Student's t test).

FIGS. 2C and 2B. Quantification of ectocalreticulin and ectoERP57 exposure after treatment with inhibitor peptides. C, treatment 4 h with BiP/PP1. D, treatment 4 h with PTD/PP1. CT26, MCA205, and EF43.fgf4 cells were untreated or treated for 4 h with Mitoxantrone, BiP, PP1, KLAK, BiP/PP1, or BiP/KLAK, followed by immunofluorescence staining with an calreticulin or ERP57-specific antibody and cytofluorometric analysis among viable (propidium iodine negative) cells.

FIG. 3. Targeted ectoERP57-inducer peptides can restore efficiently the antitumor vaccination induced by targeted proapoptotic peptides and chemotherapy. Untreated CT26, MCA205, and EF43.fgf4 cells or treated for the appropriate time with medium alone, Mitoxantrone (Mitox), mitomycin C (MC), PP1, KLAK, PTD (A, C, E), PTD/PP1 (A, C, E), DP1 (A, C, E), PTD/PP1+DP1 (A, C, E), BiP (B, D, F), BiP/PP1 (B, D, F), BiP/KLAK (B, D, F), or BiP/PP1+BiP/KLAK (B, D, F). In some groups, cells treated with MC or DP1 were followed by the incubation with recombinant calreticulin; cells treated 6 h with PTD/PP1 or BiP/PP1 were followed by the treatment for 24 h with MC; cells treated 6 h with PTD/PP1 were followed by the treatment with DP1; or cells treated with BiP/PP1 6 h were followed by the treatment with BiP/KLAK. The antitumor vaccination is measured in BALB/c mice for CT26 and EF43.fgf4 and in C57Bl/6 for MCA205. Mice were immunized subcutaneously (s.c.) with PBS or with dying treated cells in one flank and followed 1 wk later with the live tumor cells in the opposite flank. The preincubation of cells with BiP/PP1 or with PTD/PP1 was able to restore the immunogenicity of targeted preapoptotic peptides and to transform the non-immunogenic death induced by mitomycin C in immunogenic death, wherein the level of antitumor vaccination is similar to the same level observed after the restoration of ectocalreticulin by recombinant calreticulin. After that, the tumor growth was monitored. All experiments were repeated three times, each group included 14 mice by experiment. *, P<0.001 (Student's t test).

FIG. 4. The in vivo administration of targeted ectoERP57-inducer peptides improved the anticancer activity of chemotherapy and targeted proapoptotic peptides by increasing their capacity to suppress the tumor growth and to extend the lifespan of mice bearing tumors. Untreated CT26 or EF43.fgf4 tumors were established in female BALB/c mice 6 wk old (Janvier). The administration of different treatments started when mean tumor volumes reached 200 mm³ and was given slowly through the tail vein in 200 L of vehicle (DMEM). The peptides (BiP, KLAK, BiP/KLAK, BiP/PP1, or BiP/PP1+BiP/KLAK) were administered one time by week at a dose of 300 g per mouse for a period of 12 wk. For the group BiP/PP1+BiP/KLAK, 150 g of the chimeric peptide BiP/KLAK was injected 6 h after the injection of 150 g of BiP/PP1. 5-FU (fluorouracil) was given once a week at a dose 25 mg/kg for a period of 12 wk, docetaxel was given once a week at a dose 20 mg/kg for a period of 12 wk.

FIGS. 4A and B. Treatment of BALB/c mice bearing CT26 colon cancer with different treatments. A, tumors treated with BiP/PP1+BiP/KLAK are significantly smaller (Student's t test; P<0.001) than untreated tumors or treated only with BiP/KLAK, BiP/PP1 or 5-FU, as shown by differences in tumor volumes between day 1 ( ) and day 40 (). B, mice treated with BiP/PP1+BiP/KLAK (+) survived longer than untreated mice ( ) or treated with 5-FU (⋄), BiP/PP1 (), or BiP/KLAK (▴) as shown by a Kaplan-Meier survival plot (n=14 animals/group and was repeated three times; P<0.05, log-rank test).

FIGS. 4C and D. Treatment of BALB/c mice bearing EF43.fgf4 mammary cancer with different treatments. C, tumors treated with BiP/PP1+BiP/KLAK are significantly smaller (Student's t test; P<0.001) than untreated tumors or treated only with BiP/KLAK, BiP/PP1 or docetaxel, as shown by differences in tumor volumes between day 1 ( )and day 30 (). D, mice treated with BiP/PP1+BiP/KLAK (+) survived longer than untreated mice ( ) or treated with docetaxel (⋄), BiP/PP1 (), or BiP/KLAK (▴), as shown by a Kaplan-Meier survival plot (n=12 animals/group and was repeated three times; P<0.05, log-rank test).

FIG. 4E. Longevity of transgenic adenocarcinoma mouse prostate mice treated with different treatments. The treatment by peptides (ProstTarget, KLAK, PP1, ProstTarget/KLAK, ProstTarget/PP1, or ProstTarget/PP1+ProstTarget/KLAK) or by docetaxel was initiated at 12 wk of age, and male mice received once a week i.v. injections of 200 g per mouse of peptides or 25 mg/kg of docetaxel. The injections were administrated for a total of ten doses. For the group injected with ProstTarget/PP1+ProstTarget/KLAK, the injection of 100 g of ProstTarget/PP1 was initiated 6 h before the injection of 100 g of ProstTarget/KLAK. The mice in ProstTarget/PP1+ProstTarget/KLAK group survived significantly longer than the other treatment groups, as shown by a Kaplan-Meier survival plot (n=13 animals/group and was repeated three times; P<0.05, log-rank test).

FIG. 5. Ectocalreticulin is required for the immune response against tumor cells treated with ectoERP57-inducer peptides and, consequently, for a successful antitumor vaccination.

FIG. 5A-C. Antitumor vaccination depends on ectocalreticulin. Untransfected CT26 colon cancer cells or cells transfected with the calreticulin siRNA were treated with medium alone (CO), mitoxantrone (Mitox), mitomycin C (MC), BiP/KLAK, or BiP/PP1+BiP/KLAK, and then incubated (+) or not ( ) with recombinant calreticulin. The antitumor vaccination was measured in BALB/c mice immunized s.c. with PBS or with tumor-treated cells in one flank, followed 1 wk later with live tumor cells in the opposite flank. A, The depletion of ectocalreticulin suppresses the immunogenicity of mitoxantrone and BiP/PP1+BiP/KLAK. B, otherwise, the supply of recombinant calreticulin was able to restore the immunogenicity of mitoxantrone, BiP/KLAK, and BiP/PP1+BiP/PP1 (mean±SE). *, P<0.001 (Student's t test). C, CT26 subclone that was stably transfected with a siRNA-based vector against ERP57 RNA (siRNA; for RNA interference). The immunogenicity of BiP/PP1+BiP/KLAK, mitoxantrone, and inhibitor of PP1/GADD34 (tautomycin) was abolished in the CT26 ERP57 siRNA-1 clone and restored with recombinant calreticulin. The antitumor vaccination was monitored by challenging BALB/c mice with dying cells treated with BiP/PP1+BiP/KLAK, mitoxantrone, or mitomycin C plus tautomycin and, whereby optionally supplied by recombinant calreticulin, in one flank and live tumor cells in the opposite flank.

FIG. 5D-E. Ectocalreticulin dictates the priming of T cells. D, untransfected CT26 cells or those transfected with calreticulin siRNA were treated with medium alone (PBS), mitomycin C, mitoxantrone, BiP/KLAK, or BiP/PP1+BiP/KLAK, and then injected into the footpads of BALB/c mice to determine the capacity of draining lymph nodes to produce interferon-in response to dying CT26. E, the exogenous supply of recombinant calreticulin was able to restore the priming of T cells. CT26 cells lacking ectocalreticulin after depletion with siRNA calreticulin were coated with recombinant calreticulin and then inject into the footpads to assess to the production of interferon-draining lymph nodes. All experiments were repeated three times. A-C, each group includes 14 mice per experiment. D and E, each group includes three mice per experiment. *, P<0.001 (Student's t test).

FIG. 6. Ectocalreticulin mediates phagocytosis of ectoERP57-inducer peptides-treated CT26 tumor cells by dendritic cells.

FIG. 6A. correlation between ectoERP57 and phagocytosis of tumor cells by dendritic cells. CT26 tumor cells labeled with CellTracker Orange after the treatment with different treatments, peptides, or drugs were cultured for 1.5 h with dendritic cells-expressed CD11c. The percentage of dendritic cell taking up tumor cells was determined by gating on double-positive dendritic cell for CellTracker Orange and for CD11c-FITC and correlated with ERP57 surface exposure.

FIG. 6B. Neutralizing of ectocalreticulin inhibited dendritic cell-mediated phagocytosis. Mitoxantrone (Mitox) or BiP/PP1+BiP/KLAK-treated or control cells (CO) were incubated with a chicken blocking calreticulin antibody before their coculture with dendritic cells. The percentage of phagocytosis was determined as in A.

FIG. 6C. Knockdown of calreticulin inhibits dendritic cell-mediated phagocytosis, but the addition of recombinant calreticulin restores phagocytosis in vitro. In CT26 transfected with calreticulin siRNA and optionally treated with recombinant calreticulin, I measured the phagocytosis of tumor cells by dendritic cell. The phagocytotic index consists in the ratio between values obtained with control cells and those obtained after mitoxantrone or BiP/PP1+BiP/KLAK treatment (with or without siRNA calreticulin and with or without recombinant calreticulin). Results are triplicates from one experiment (mean±SD) and representative of three independent experiments. *, P<0.001 (Student's t test).

### DETAILED DESCRIPTION OF PREFERRED EMBODIMENTS

### Materials and Methods

### Design and Synthesis of Peptides.

The inhibitor peptide of the PP1/GADD34 complex consists of an amino acid fragment of GADD34. Several peptide domains (PTD, BiP, ProstTarget, KLAK, and PP1) and chimeric peptides [PTD/PP1, BiP/PP1, ProstTarget/PP1, PTD/KLAK (DP1), BiP/KLAK, ProstTarget/KLAK] were synthesized and then purified by reversed phase high-performance liquid chromatography to >90% purity and confirmed by mass spectrometry. Peptides were reconstituted in water for in vitro and in vivo use. The amino acids sequence of the protein transduction domain-5 peptide termed “PTD” is “RRQRRTSKLMKR” where the letters in this sequence are associated with the following designations: R=Arg, Q=Gln, T=Thr, S=Ser, K=Lys, L=Leu, M=Met, (Sequence 1, ID NO. 1-12). The amino acids sequence of the tumor antigen BiP/GRP78 peptide termed “BiP” is “WIFPWIQL” where the letters in this sequence are associated with the following designations: W=Trp, I=Ile, F=Phe, P=Pro, Q=Gln, L=Leu, (Sequence 2, ID NO. 1-8). The amino acids sequence of ProstTarget peptide termed “ProstTarget is “SMSIARL” where the letters in this sequence are associated with the following designations: S=Ser, M=Met, I=Ile, A=Ala, R=Arg, L=Leu, (Sequence 3, ID NO. 1-7). The killer peptide termed “KLAK” is the proapoptotic peptide D (KLAKLAK)₂ with an amphipatic helix where the letters in this sequence are associated with the following designations: K=Lys, L=Leu, A=Ala, (Sequence 4, ID NO. 1-14). The amino acids sequence of the inhibitor peptide of the interaction between PP1 and GADD34 termed “PP1” is “LKARKVRFSEKV” where the letters in this sequence are associated with the following designations: L=Leu, K=Lys, A=Ala, R=Arg, V=Val, F=Phe, S=Ser, E=Glu, (Sequence 5, ID NO. 1-12).

### Antitumor Vaccination and Intratumoral Injection.

All animals were bred and maintained according to the Federation of European Laboratory Animal Science Associations and the Animal Experimental Ethics Committee Guidelines. CT26 were treated for 30 min with the following peptides: KLAK (10 mol/L), PP1 (10 mol/L), PTD (10 mol/L), PTD/PP1 (10 mol/L), and DP1 (10 mol/L), or KLAK (15 mol/L), PP1 (15 mol/L), BiP (15 mol/L), BiP/PP1 (15 mol/L), and BiP/KLAK (15 mol/L). EF43.fgf4 was treated for 30 min with the following peptides: KLAK (10 mol/L), PP1 (10 mol/L), PTD (10 mol/L), PTD/PP1 (10 mol/L), and DP1 (20 mol/L), or KLAK (20 mol/L), PP1 (15 mol/L), BiP (20 mol/L), BiP/PP1 (15 mol/L), and BiP/KLAK (20 mol/L). MCA205 was treated for 30 min with the following peptides: KLAK (50 mol/L), PP1 (10 mol/L), PTD (50 mol/L), PTD/PP1 (10 mol/L), and DP1 (50 mol/L), or KLAK (70 mol/L), PP1 (15 mol/L), BiP (70 mol/L), BiP/PP1 (15 mol/L), and BiP/KLAK (70 mol/L). In addition, for the groups wherein mice were vaccinated with cells treated with PTD/PP1+DP1, BiP/PP1+BiP/KALAK, MC+PTD/ PP1, or MC+BiP/PP1, cells were treated 6 h with PTD/PP1 or BiP/PP1 before the treatment with MC, DP1, or BiP/KALAK (because in my previous works, I have shown that ectocalreticulin should be given many hours before the manifestation of any apoptotic signs). In some conditions, cells were treated for 4 h with Mitox (1 mol/L) and 24 h with MC (30 mol/L) with or without recombinant calreticulin (45 g), and in some groups, cells were treated with DP1 or BiP/KLAK, followed by the addition of recombinant calreticulin (45 g). Treated CT26 or EF43.fgf4 cells (3×10⁶) were inoculated s.c. in 200 mL of PBS into BALB/c 6-wk-old female mice (wild type or nu/nu for some experiments done with CT26; Janvier), into the lower flank, whereas 5×10⁵ untreated control cells were inoculated into the opposite flank. In contrast, 25×10⁴ treated MCA205 cells were inoculated s.c. in 200 mL of PBS into C57Bl/6 6-wk-old mice (Janvier), into the lower flank, whereas 30×10⁴ untreated control cells were inoculated into the opposite flank. To assess the specificity of the immune response against CT26, EF43.fgf4, or MCA205, I injected either 5×10⁶ of CT26 or EF43.fgf4 or 3×10⁵ of MCA205. Tumors were evaluated twice weekly using a caliper. In a series of experiments, wild-type BALB/c carrying palpable CT26 or EF43.fgf4 tumors received one intratumoral injection once a week for 12 wk of 100 mol/L PBS with same protocol, time injection, and kinetics as those used for antitumor vaccination, and containing the same concentration of each peptide as those used in vitro, as well as for recombinant calreticulin (45 g), 5-fluorouracil (5-FU; 25 mg/kg; Sigma), or docetaxel (20 mg/kg; Aventis). None of these treatments caused macroscopic necrosis. In some mice, either T cells (CD4⁺ or CD8⁺) were depleted by i.p. injection of the purified monoclonal antibodies YTS191 (100 mg) and H35.17.2 (250 mg; obtained from American Type Culture Collection), or natural killer (NK) cells were depleted by i.p. of antiasialo GM1monoclonal antibody (250 mg; Wako Bioproducts), respectively, 4 d before challenge with treated tumor cells and 3 d before injection of live tumor cells (inoculated 1 wk after that of the treated cells).

### Assessment of Local Immune Responses

CT26 cells (5×10⁵) treated with the appropriate product and for an adequate time, as used for the antitumor vaccination, were injected into the footpad of BALB/c mice. Five days later, popliteal lymph node cells were recovered by homogenizing and filtering the organ through a sterile cell strainer (70 m; BD Biosciences). Lymph node cells (1×10⁵) were cultured in 200 L of complete culture medium in the presence or absence of CT26 cell lysates killed by a freeze-thaw cycle in round-bottom, 96-well plates. Three days later, the supernatants were harvested, and interferon-secretion was determined by ELISA (BD Biosciences). In one series of experiments, CD11c-GFP DT Kb mice (10) were injected i.p. with 100 ng diphteric toxin (or PBS as a vehicle control) on the same day as tumor-treated cells were injected into the food pad.

### In Vivo Administration of Peptides and Chemotherapy

BALB/c carrying palpable CT26 or EF43.fgf4 tumors (seeded with 3×10⁵ cells) were divided into groups (n=12 for EF43.fgf4 and 14 for CT26), and treatments started when mean tumor volumes reached 200 mm³. Six constructions of peptides (BiP, KLAK, BiP/KLAK, BiP/PP1, or BiP/PP1+BiP/KLAK) were injected i.v. in BALB/c bearing CT26 tumors or EF43.fgf4. Therapeutic and control peptides were systemically administered (tail vein) at 300 g per dose per mouse in 200 L of vehicle (DMEM) weekly for a period of 12 wk. For the group BiP/PP1+BiP/KLAK, 150 g of the chimeric peptide BiP/KLAK was injected 6 h after the injection of 150 g of BiP/PP1. In some groups, 5-FU (25 mg/kg) or docetaxel (20 mg/kg) was injected weekly for 12 wk. Tumor volumes were measured twice a week, as described previously. For male transgenic adenocarcinoma mouse prostate mice (Charles River), the i.v. injection of peptides 200 g per mice per week (13 mice per group; ProstTarget, KLAK, PP1, ProstTarget/KLAK, ProstTarget/PP1, or ProstTarget/KLAK+ProstTarget/PP1) or the injection of docetaxel (20 mg/kg; Aventis) was initiated at 12 wk of age for 10 wk. For the group ProstTarget/KLAK+ProstTarget/PP1, 100 g of ProstTarget/KLAK was injected 6 h after the injection of 100 g of ProstTarget/PP1. After that, the effect of the different treatments on the longevity of transgenic adenocarcinoma mouse prostate mice was measured.

### Statistical Analyses

Experimental results are expressed as mean±SEs of triplicate plates. Statistical significance was determined by Student's t tests or by log rank test. For Student's t tests, the statistical significance level was set at P<0.001, and for log rank tests, the statistical significance level was set at P<0.05.

The following examples provide some illustrations of the present invention.

### EXAMPLE 1

### Design of Chimeric Targeted Peptides.

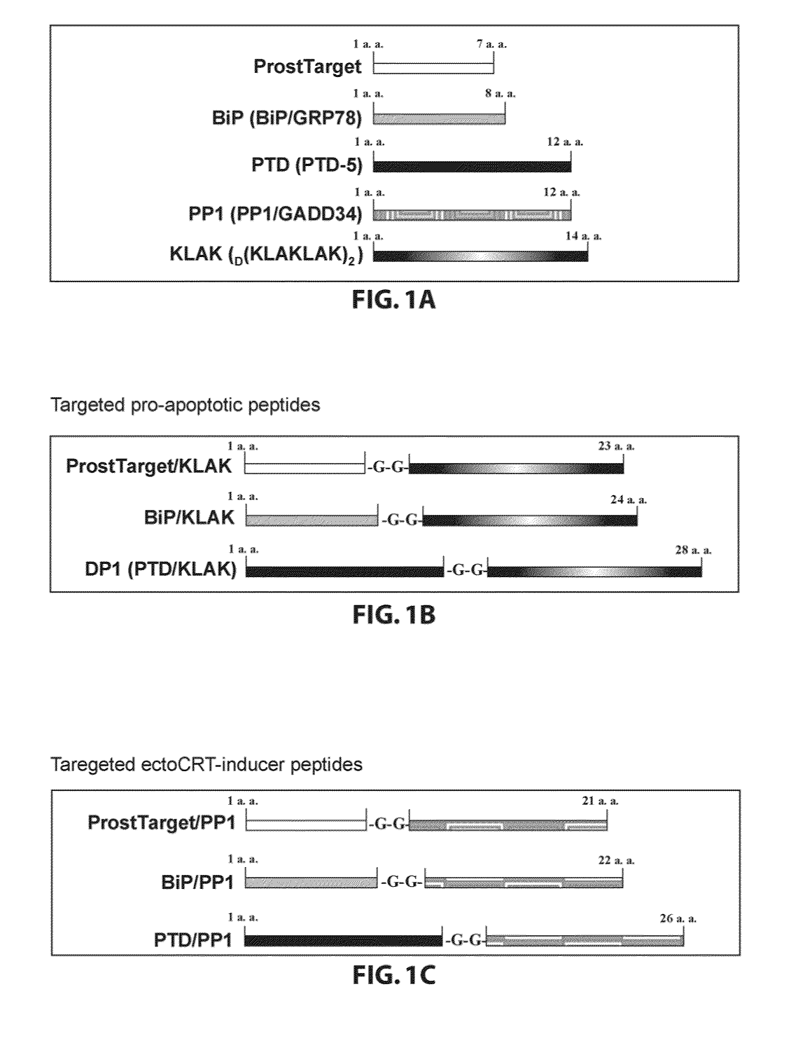

The following peptide domains have been used: two targeting peptides, one killer (proapoptotic) peptide, one inhibitor peptide, and one protein transduction domain-5 peptide (FIG. 1). The chimeric peptides have been designed to create two families of targeted peptides: the targeted proapoptotic peptides and the targeted ectoERP57-inducer peptides. The two tumor targeted peptides are BiP/GRP78-targeting peptide and ProstTarget peptide (FIG. 1A). BiP/GRP78 peptide (WIFPWIQL) termed “BiP” targets the tumor antigen BiP/GRP78 in vitro and in vivo and targets tumor cells specifically in vivo and in human cancer specimens ex vivo. ProstTarget peptide (SMSIARL) termed “ProstTarget” targets the prostate of transgenic adenocarcinoma mouse prostate mice through vasculature address after i.v. administration and delivers several biologically active compounds to the prostate cells. The killer peptide termed “KLAK” is the proapoptotic peptide_{D} (KLAKLAK)₂ with an amphipatic helix to avoid degradation by proteases, forming antimicrobial peptide that preferentially disrupts eukaryotic mitochondrial membranes upon receptor-mediated internalization and was used to be nontoxic outside cells but toxic when internalized into targeted cells (FIG. 1A). The inhibitor peptide (LKARKVRFSEKV) of the complex PP1/GADD34 termed “PP1” consists of an amino acid fragment of GADD34 and was designed to inhibit the interaction between PP1 and GADD34 to induce ectocalreticulin and ectoERP57 (FIG. 1A). The protein transduction domain-5 peptide (RRQRRTSKLMKR) termed “PTD” transfer into the cells therapeutic proteins and DNA and can facilitate rapid internalization of a variety of cargos in vitro and in vivo in a large variety of cell lines and solid tumors (FIG. 1A). The family of targeted ectoERP57-inducer peptides contains three members: BiP/PP1, PTD/PP1, and ProstTarget/PPI. The first member is the chimeric peptide BiP/PP1, which is composed of BiP peptide coupled to PP1 inhibitor peptide, separated by diglyicine spacer, to generate the BiP/PP1 peptide (WIFPWIQLGGLKARKVRFSEKV; FIG. 1B). The objective of synthesizing this peptide was to create an in vivo and in vitro tumor-targeting peptide that induces selectively the immunogenic signature, ectocalreticulin and ectoERP57, on the cell surface of tumor cells. BiP/PP1 will be used for antitumor vaccination, systemic administration (i.v.), and intratumor injection. BiP/PP1 could be functional in vitro, in vivo, and intratumor use. The second chimeric peptide is composed of PTD peptide coupled to PP1, separated by diglyicine spacer, to generate the PTD/PP1 peptide (RRQRRTSKLMKRGGLKARKVRFSEKV; FIG. 1B). The objective of synthesizing this peptide was to create an in vitro cargo that can internalize efficiently the inhibitor peptide of the PP1/GADD34 complex into the cell to induce the immunogenic signature. PTD/PP1 could be functional in vitro and for intratumor use. The third chimeric peptide was composed of ProstTarget peptide coupled to PP1 inhibitor peptide, separated by diglyicine spacer, to generate the ProstTarget/PP1 peptide (SMSIARLGGLKARKVRFSEKV; FIG. 1 B). The objective of synthesizing this peptide was to create an in vivo tumor-targeting peptide that induces selectively the immunogenic signature, ectocalreticulin and ectoERP57, on the cell surface of prostate cancer cells of the transgenic adenocarcinoma mouse prostate mice. ProstTarget/PP1 is for in vivo use. The family of targeted proapoptotic peptides is composed of three members: BiP/KLAK, PTD/KLAK, and ProstTarget/KLAK. The first member is a chimeric peptide composed of BiP peptide coupled to antimicrobial proapoptotic peptide KLAK_{D} (KLAKLAKKLAKLAK), separated by diglyicine spacer, to generate the BiP/KLAK peptide (WIFPWIQLGGKLAKLAKKLAKLAK; FIG. 1B). The objective of synthesizing this peptide was to create an in vitro and in vivo killer peptide that can kill selectively the tumor cells in vivo and in vitro. BiP/KLAK could be functional in vitro, in vivo, and in intratumor injection. The second is a chimeric peptide composed of PTD peptide coupled to antimicrobial proapoptotic peptide KLAK_{D} (KLAKLAKKLAKLAK), separated by diglyicine spacer, to generate the proapoptotic peptide “DP1” (RRQRRTSKLMKRGGKLAKLAKKLAKLAK; FIG. 1B). The objective of synthesizing this peptide was to create an in vitro cargo that can internalize efficiently the killer peptide into eukaryotic cells. Consequently, this peptide is toxic only into the cells and nontoxic outside of the cell. This peptide DP1 could be functional in vitro use and for intratumor injection. The third chimeric proapoptotic peptide was composed of ProstTarget peptide (SMSIARL) coupled to antimicrobial proapoptotic peptide KLAK_{D} (KLAKLAKKLAKLAK), separated by diglyicine spacer, to generate the ProstTarget/KLAK peptide (SMSIARLGGKLAKLAKKLAKLAK; FIG. 1B). The objective of synthesizing this peptide was to create in vivo proapoptotic peptide that can kill selectively prostate tumor cells in transgenic adenocarcinoma mouse prostate mice. ProstTarget/KLAK will be used for systemic administration by i.v. injection.

### EXAMPLE 2

Inhibitor Peptides of PP1/GADD34 Complex Inhibit Interaction between PP1 and GADD34 and Induce Ectocalreticulin and EctoERP57.

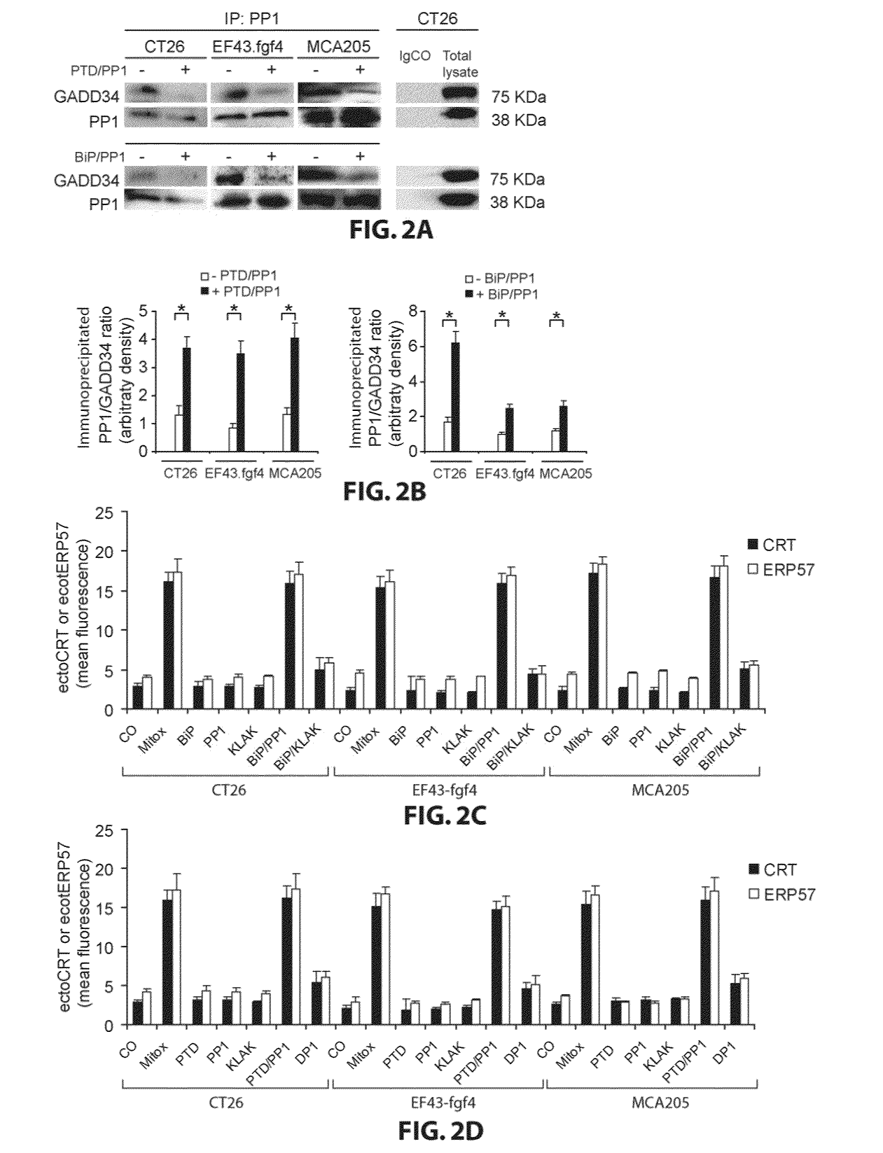

We have recently reported that the cell surface exposure of ectocalreticulin and ectoERP57 was induced by the inhibition of the interaction between PP1 and GADD34. EctoERP57 is cotranslocated to the cell surface with ectocalreticulin in the same molecular complex. To confirm that PTD/PP1 and BiP/PP1 inhibits PP1/GADD34 complex, I have done coimmunoprecipitation experiments on untreated CT26, MCA205, or EF43.fgf4 or treated for 4 h either with PTD/PP1 or BiP/PP1. The concentrations and times used in vitro for the treatment of cells with the different PP1/GADD34 inhibitor peptides were established in preliminary dose-response experiments designed to induce the maximum level of ectocalreticulin and ectoERP57 (data not shown). Immunoprecipitation of PP1 confirms the interaction with GADD34 in untreated CT26, MCA205, or EF43.fgf4 cells (FIG. 2A). The amounts of interaction between PP1 and GADD34 were largely inhibited in CT26, MCA205, or EF43.fgf4 cells after the treatment with PTD/PP1 or BiP/PP1 (FIGS. 2A and B). These data show that BiP/PP1 and PTD/PP1 can inhibit largely the interaction between PP1 and GADD34. Because the cell surface exposure of calreticulin and ERP57 is conditioned by the inhibition of the complex PP1/GADD34, I checked whether the inhibition by PTD/PP1 or BiP/PP1 could induce ectocalreticulin and ectoERP57. The surface exposure of calreticulin and ERP57 in CT26, MCA205, and EF43.fgf4 cells was confirmed by cytofluorometric analysis after BiP/PP1 (FIG. 2C) or PTD/PP1 (FIG. 2D) treatment. The cytofluorometric Analysis was done only among live cells (propidium iodine negative). In these experiments, I verify the specificity of PP1 peptides, which are only functional when coupled to the translocation domain BiP or PTD. Without this domain, PP1 is incapable to be translocated into the cell and, consequently, not able to induce ectocalreticulin and ectoERP57. Similarly, the translocation domains BiP and PTD are not able to induce ectocalreticulin or ectoERP57 without the inhibitor peptide PP1 (FIG. 2C and D). The level of ectocalreticulin and ectoERP57 induced by BiP/PP1 or PTD/PP1 was identical to the level induced by immunogenic treatment such as Mitoxantrone Mitox (FIGS. 2C and D) and to the historical level obtained in our previous work with mitoxantrone or with chemical inhibitors of PP1/GADD34 such as tautomycine, calyculine A, and salubrinal. In addition, I observed that the treatment with the two chimeric proapoptotic peptides BiP/KLAK or DP1 is not accompanied by an important level of ectocalreticulin or ectoERP57. This level is identical to the level obtained with nonimmunogenic treatments such as mitomycin c detailed in our previous work.

### EXAMPLE 3

### Cell Death Induced by Targeted Proapoptotic Peptides DP1 and BiP/KLAK.

KLAK peptide domain is known to be toxic inside the cell and nontoxic outside of the cell. Consequently, KLAK could not function as proapoptotic peptide if it is not coupled to a transduction domain either BiP or PTD. To examine this particularity of DP1 (PTD/KLAK) and BiP/KLAK to induce cell death in contrast to PTD, KLAK, and BiP peptide domains, the cell viability was determined. CT26, MCA205, or EF43.fgf4 cells were treated for 4 h with BiP, KLAK, PTD, DP1, or BiP/KLAK, and the amount of apoptosis was determined. Induction of apoptosis was a quick process, with observable changes in cell morphology as early as 25 minutes after the administration of DP1 or BiP/KLAK to cells in vitro. In contrast, neither the mitochondrial disruption domain alone (KLAK) nor the transduction domain alone (PTD or BiP) significantly affected cells viability (Supplementary FIG. S1). DP1 and BiP/KLAK were able to induce a very significant reduction in the viability of CT26 (Supplementary FIGS. S1A and B), EF43.fgf4 (Supplementary FIGS. S1C and D), and MCA205 (Supplementary FIGS. S1E and F) in contrast to the negligible response observed in KLAK, BiP, or PTD treated cells.

### EXAMPLE 4

Antitumor Vaccination Induced by Targeted Proapoptotic Peptides and Chemotherapy is Highly Improved after the Intensification of Ectocalreticulin Mediated by Targeted EctoERP57-Inducer Peptides.

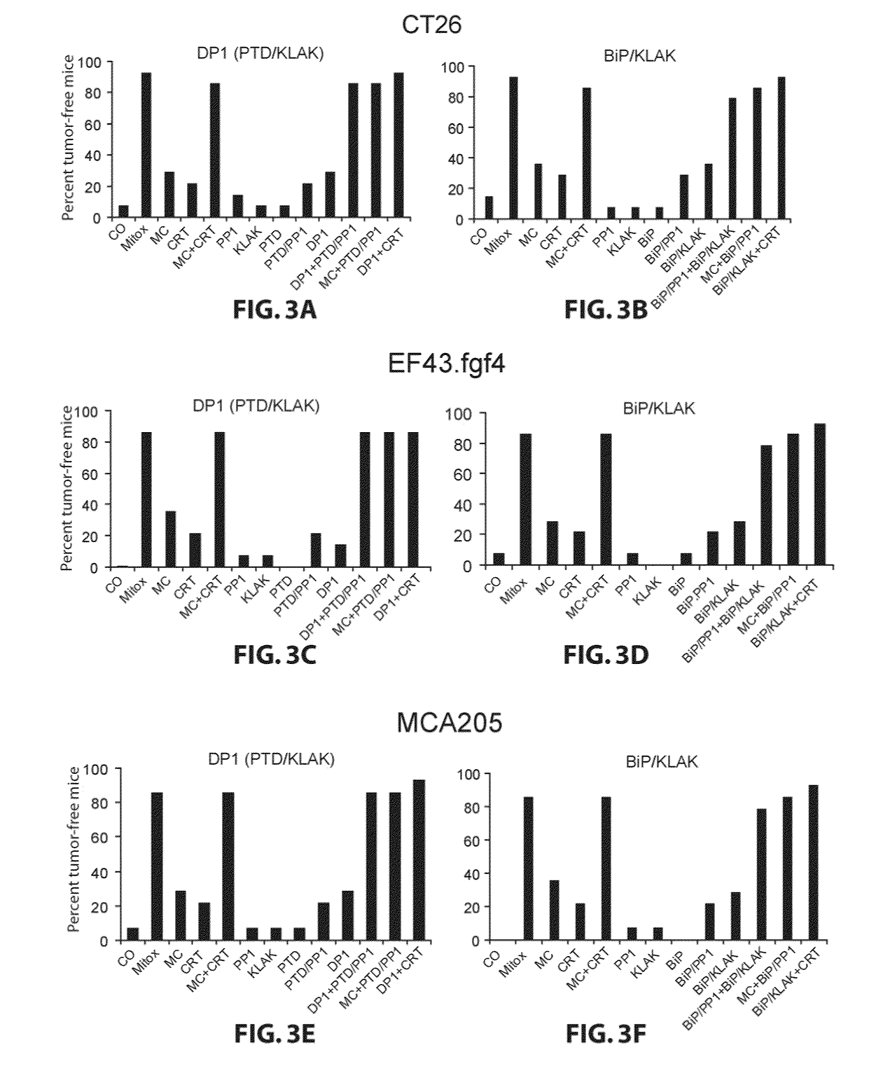

Because (a) I showed in FIG. 2 that proapoptotic peptides DP1 and BiP/KLAK induce a weak level of ectocalreticulin and ectoERP57 compared with the level obtained with immunogenic inducers (e.g., Mitoxantrone) or with PP1/GADD34 inhibitor peptides (e.g., BiP/PP1 and PTD/PP1); (b) and I showed in the Supplementary FIG. S1 that proapoptotic peptides DP1 and BiP/KLAK are very potent apoptosis inducer in several cells lines (CT26 colon cancer, MCA205 fibrosarcomas, and EF43.fgf4 mammary carcinoma), I decided to check whether the antitumor vaccination induced by proapoptotic peptides would be improved by increasing the level of ectocalreticulin on CT26, MCA205, or EF43.fgf4-treated cells. The weak level of ectocalreticulin and ectoERP57 induced by DP1 or BiP/KLAK was very similar to the level induced by nonimmunogenic treatment such as MC and very less than the level induced by immunogenic treatment such as Mitox or the inhibitor peptides PTD/PP1 or BiP/PP1 (FIG. 2; Supplementary FIG. S2). The inhibitor peptides BIP/PP1 and PTD/PP1 were able to restore the surface translocation of calreticulin and ERP57 in DP1 or BiP/KLAK-treated cells to a level equivalent to the one obtained in cells treated with immunogenic inducers (e.g., Mitox) or with the recombinant calreticulin (Supplementary FIG. S2). However, I have confirmed my previous results obtained in precedent works on the capacity of recombinant calreticulin to restore the immunogenicity of nonimmunogenic cell death such as death induced by MC (FIG. 3). The weak level of ectocalreticulin induced by DP1 and BiP/KLAK correlated with a weak level of antitumor vaccination (FIG. 3). The low level of immunogenicity induced in DP1 or BiP/KLAK-treated cells was identical to the one induced by a classical nonimmunogenic cell death inducers such as mitomycin MC (FIG. 3). Having shown in Supplementary FIG. S2 the capacity of the inhibitor peptides BiP/PP1 and PTD/PP1 to restore the level of ectocalreticulin and ectoERP57 in cells treated with BiP/KLAK, DP1, or MC, I decided to check whether the restoration of the level of ectocERP57 could also improve the immunogenicity obtained by MC, BiP/KLAK, or DP1. As expected, the fact that I have restored the level of ectocERP57 by treating the cells with PTD/PP1 or BiP/PP1 6 hours before the treatment with DP1, BiP/KLAK, or MC, I was able to restore the immunogenicity to a level identical to the one obtained after an ectocalreticulin restoration with recombinant calreticulin, such as in MC plus recombinant calreticulin or DP1 plus recombinant calreticulin, or to the one obtained after immunogenic treatment such as Mitox (FIG. 3; I note that I have treated the cells with PTD/PP1 or BiP/PP1 6 hours before the treatment with DP1, BiP/KLAK, or MC because, in my previous works, I showed that ectocalreticulin occurs many hours before the manifestation of any apoptosis signs). In conclusion, the addition of ectoERP57-inducer peptides was also able to restore the immunogenicity of both proapoptotic peptides such as DP1 or BiP/PP1 and chemotherapy such as MC: to a level similar to the one obtained after the addition of recombinant calreticulin. In summary, the fact that I restored the level of ectocalreticulin by adding the inhibitor peptides BiP/PP1 or PTD/PP1 (or by recombinant calreticulin), I was able to improve highly the antitumor vaccination obtained by proapoptotic peptides or by chemotherapy. In other terms, the efficiency of antitumor vaccination obtained by proapoptotic peptides or by chemotherapy-treated cells correlated with the level of ectoERP57 (Supplementary FIG. S2 and FIG. 3).

### EXAMPLE 5

### The In Vivo Anticancer Activity of Targeted Proapoptotic Peptides is Highly Improved by the Addition of Targeted EctoERP57-Inducer Peptides.

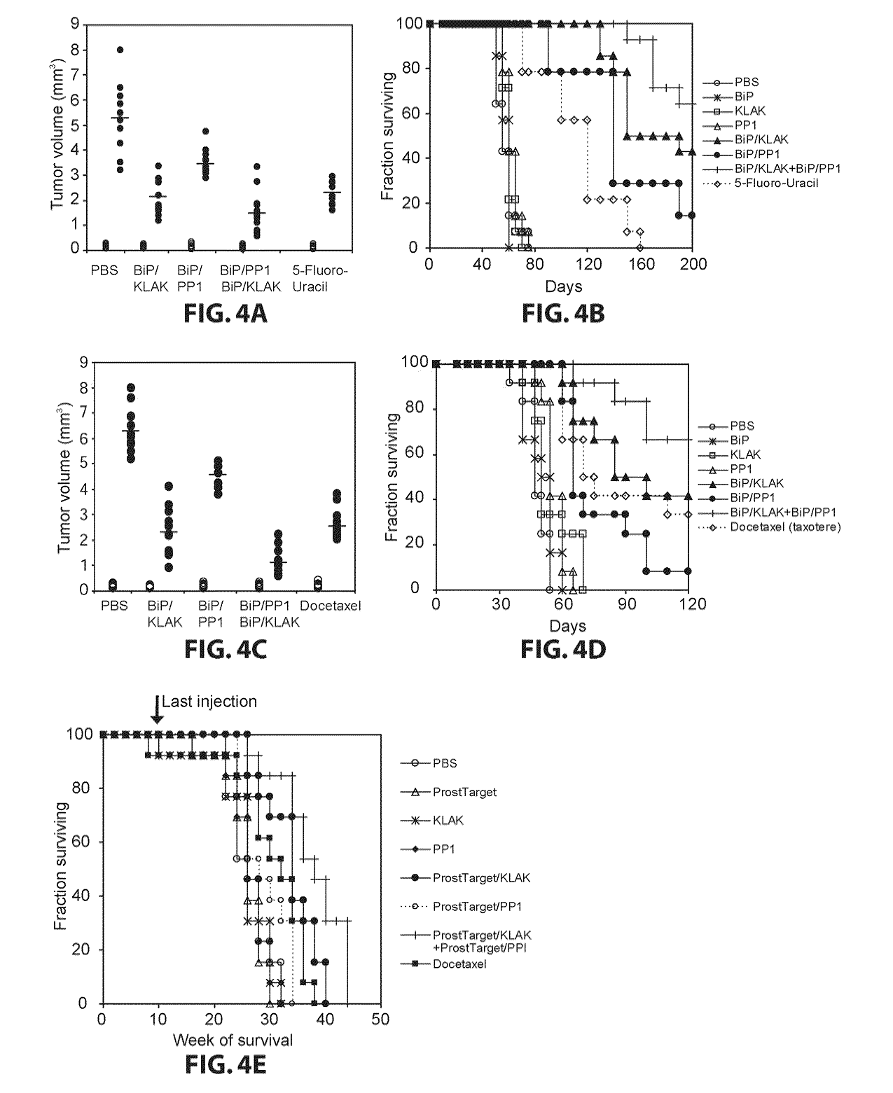

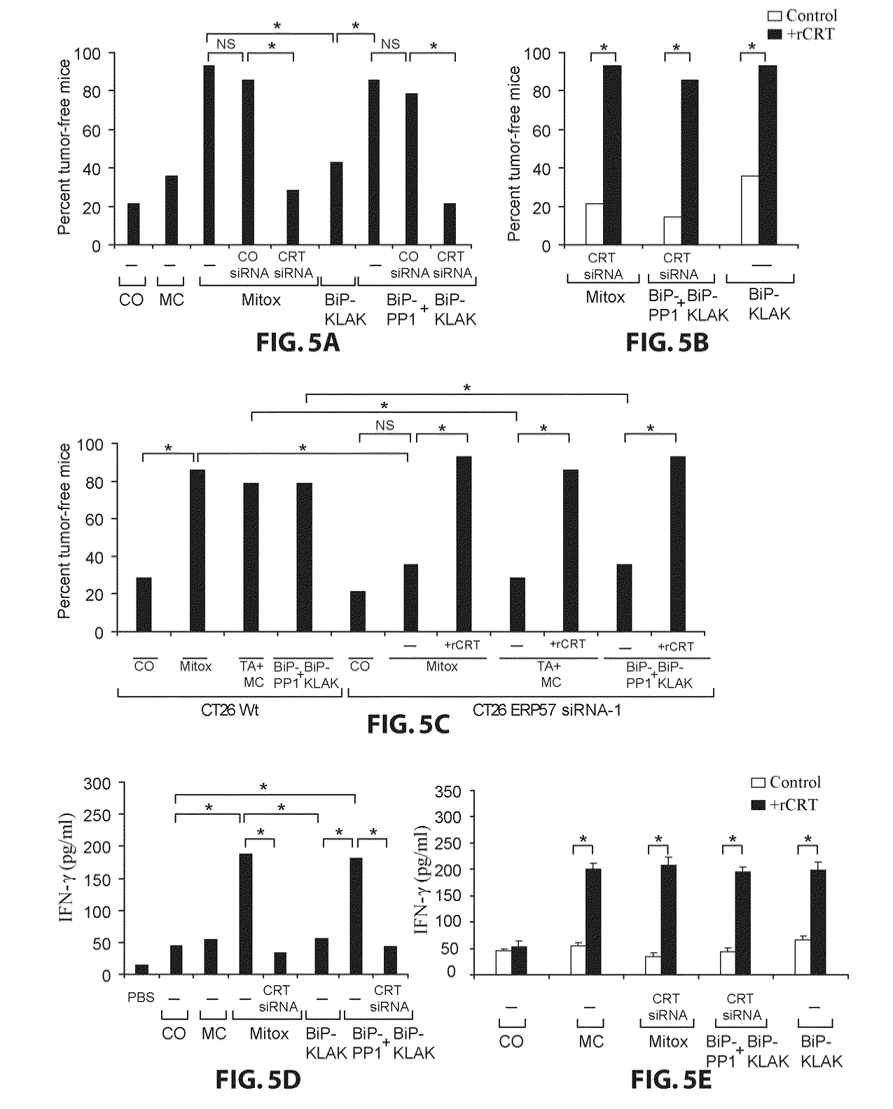

Given the results obtained in FIGS. 2 and 3, which show that the antitumor vaccination efficiency of targeted proapoptotic peptides and chemotherapy depended largely on the quantity of ectoERP57, I aimed to evaluate the exact role of ectoERP57 in the anticancer activity of targeted proapoptotic peptides. First, I proceeded to test the in vivo anticancer effect of the ectoERP57-inducer peptides using immunocompetent BALB/c mice bearing CT26 colon cancer or EF43.fgf4 mammary carcinoma. At day 40, the posttreatment mean tumor volume in the groups BALB/c bearing CT26 tumors treated with BiP/KLAK plus BiP/PP1 represented 28% of that observed in control groups, 71% of that observed with BiP/KLAK, 44% of that observed with BiP/PP1, and 64% of that observed with 5-FU, a chemotherapy compounds used classically in clinics to treat colon cancer (FIG. 4A). At day 200, 64% of mice bearing CT26 tumors treated with BiP/KLAK plus BiP/PP1 survived compared with 43% observed with BiP/KLAK, with 14% observed with BiP/PP1, and with 0% observed with 5-FU (FIG. 4B). In addition, the same profile of antitumor activity was observed in second tumor model consisting of BALB/c bearing EF43.fgf4 tumors. At day 30, the posttreatment mean tumor volume in the groups BALB/c bearing EF43.fgf4 tumors treated with BiP/KLAK plus BiP/PP1 represented 19% of that observed in control groups, 49% of that observed with BiP/KLAK, 32% of that observed with BiP/PP1, and 45% of that observed with docetaxel (taxotere), a chemotherapy compounds used classically in clinics to treat mammary cancer (FIG. 4C). At day 120, 67% of mice bearing EF43.fgf4 tumors treated with BiP/KLAK plus BiP/PP1 survived compared with 42% observed with BiP/KLAK, with 8% observed with BiP/PP1, and with 33% observed with docetaxel (FIG. 4D). Second, the intratumor injection of BiP/PP1 that followed 6 hours later by the injection of proapoptotic peptides BiP-KLAK provoked (a) a significant suppression of tumor growth compared with the injection of only BiP/KLAK or BiP/PP1, or to chemotherapy (5-FU or docetaxel); and (b) an important augmentation in the longevity observed with BiP/PP1+BiP/KLAK compared with BiP/KLAK, BiP/PP1, or chemotherapy drugs (5-FU or docetaxel; Supplementary FIG. S3). In addition, I have analyzed the effect of the inhibitor peptides after i.v. administration on the longevity of transgenic adenocarcinoma mouse prostate mice. I have observed a significant increase in the surviving rate of transgenic adenocarcinoma mouse prostate mice treated with ProstTarget/PP1, followed 6 hours later by ProstTarget/KLAK, compared with the treatment with ProstTarget/KLAK alone, ProstTarget/PP1 alone, or the classic chemotherapy (docetaxel; FIG. 5). Altogether, these results showed clearly that the intensification of ectoERP57 by the addition of the PP1/GADD34 inhibitor peptides mediates (a) an important suppression in the tumor growth and (b) a significant augmentation in mice longevity compared with BiP/KLAK alone or to the classic chemotherapy.

### EXAMPLE 5

### Requirement of EctoERP57 for the Immunogenicity of Tumor Cells Induced by EctoERP57-Inducer Peptides.

Because we have recently reported that ectocalreticulin dictates the immunogenicity of tumor cells treated by anthracyline, UVC, or irradiation, I aimed to evaluate whether the restoration of the immunogenicity by ectoERP57-inducer peptides is related also to the augmentation in ectocalreticulin quantity. First, after the depletion of ectoERP57 by calreticulin siRNA, the immunogenicity of BiP/PP1+BiP/KLAK or mitoxantrone-treated CT26 cells was abolished (FIG. 5A). This immunogenicity was restored when recombinant calreticulin was used to complement the ectocalreticulin defect induced by calreticulin siRNA (FIG. 5B). Nevertheless, the immunogenicity observed with BiP/KLAK was equivalent to the one observed with nonimmunogenic cell death inducers such as MC (FIG. 5A). Otherwise, the augmentation of ectocalreticulin by a direct supply of recombinant calreticulin or after the stimulation with EctoERP57-inducer peptides restores identically the immunogenicity of tumor cells treated only with BiP/KLAK (FIG. 5B). Second, I evaluated the immunogenicity of ectoERP57-inducer peptides in CT26 siRNA-1 ERP57 cells (1), clone with stable expression of ERP57 siRNA vector, wherein surface cell exposure of calreticulin after mitoxantrone or PP1/GADD34 chemical inhibitors (e.g., tautomycin) treatment was specifically abolished (1). Therefore, I decided to verify by another way the effect of the inhibition of ectoERP57 on the immunogenicity induced by BiP/PP1+BiP/KLAK-treated cells. Indeed, I obtained the same result as that presented in FIGS. 5A and B by a suppression of the antitumor vaccination after BiP/PP1+BiP/KLAK, mitoxantrone, or mitomycin C plus tautomycin treatment (FIG. 5C). The defect in the immunogenicity observed in the stable clone treated with BiP/PP1+BiP/KLAK, mitoxantrone, or mitomycin C plus tautomycin was restored after the addition of recombinant calreticulin (FIG. 5C). Then, I evaluated the role of ectoERP57 in the production of interferon-by popliteal lymph nodes. Similarly, I found that ectoERP57 is crucial for the secretion of interferon-by popliteal lymph nodes (FIGS. 5D and E). The secretion of interferon-was very important with cells treated by mitoxantrone or by BiP/PP1+BiP/KLAK (FIG. 5D) in contrast to the low level observed by nonimmunogenic treatment such as mitomycin C or BiP/KLAK (FIG. 5D). The depletion of ectoERP57 by calreticulin siRNA abolishes the production of interferon-after the treatment with mitoxantrone or with BiP/PP1+BiP/KLAK (FIG. 5D). In addition, the supply of recombinant calreticulin had the capacity to restore interferon-secretion after mitoxantrone or BiP/PP1+BiP/KLAK treatment (FIG. 5E). Similarly, the supply of recombinant calreticulin had the potential to restore the production of interferon-after the injection of tumor cells treated with nonimmunogenic treatment such as mitomycin C or BiP/KLAK (FIG. 5E). In summary, the addition of recombinant calreticulin restored interferon-secretion (FIGS. 5D and E) and the immunogenicity (FIG. 5B) of nonimmunogenic treatment such as that of mitomycin C and BiP/KLAK. Thus, ectoERP57 determines the immunogenicity of cancer cell death induced by ectoERP57-inducer peptides.

### EXAMPLE 6

### Requirement of EctoERP57 to Mediate Phaqocytosis of EctoERP57-Inducer Peptides—Treated Tumor Cells by Dendritic Cells.

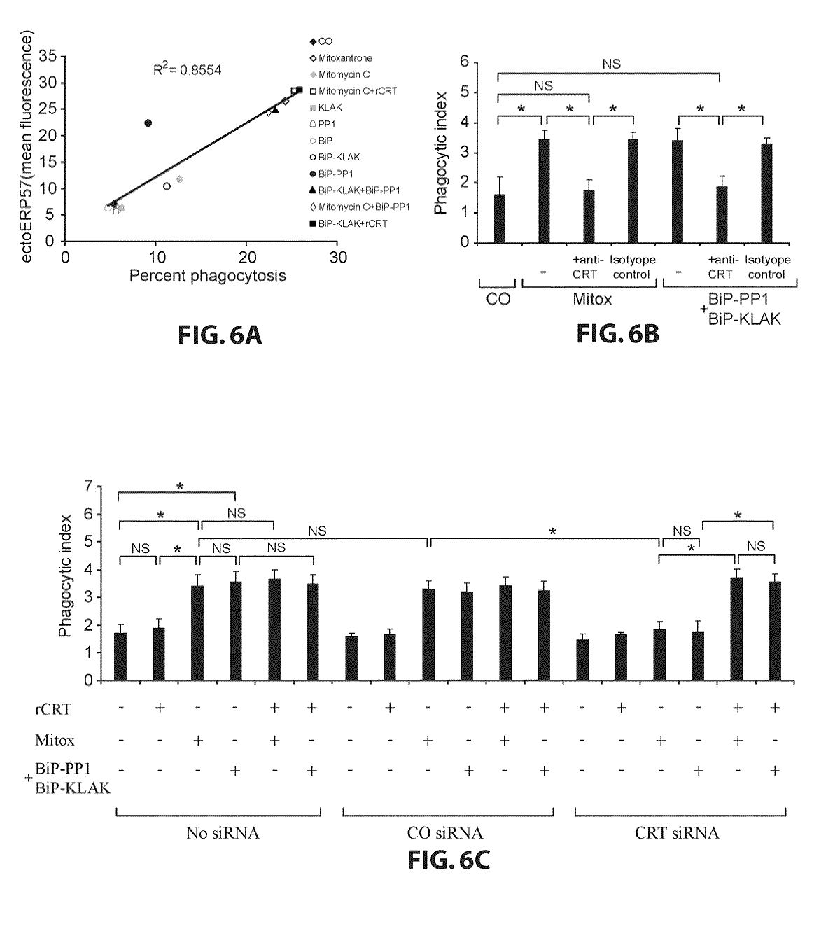

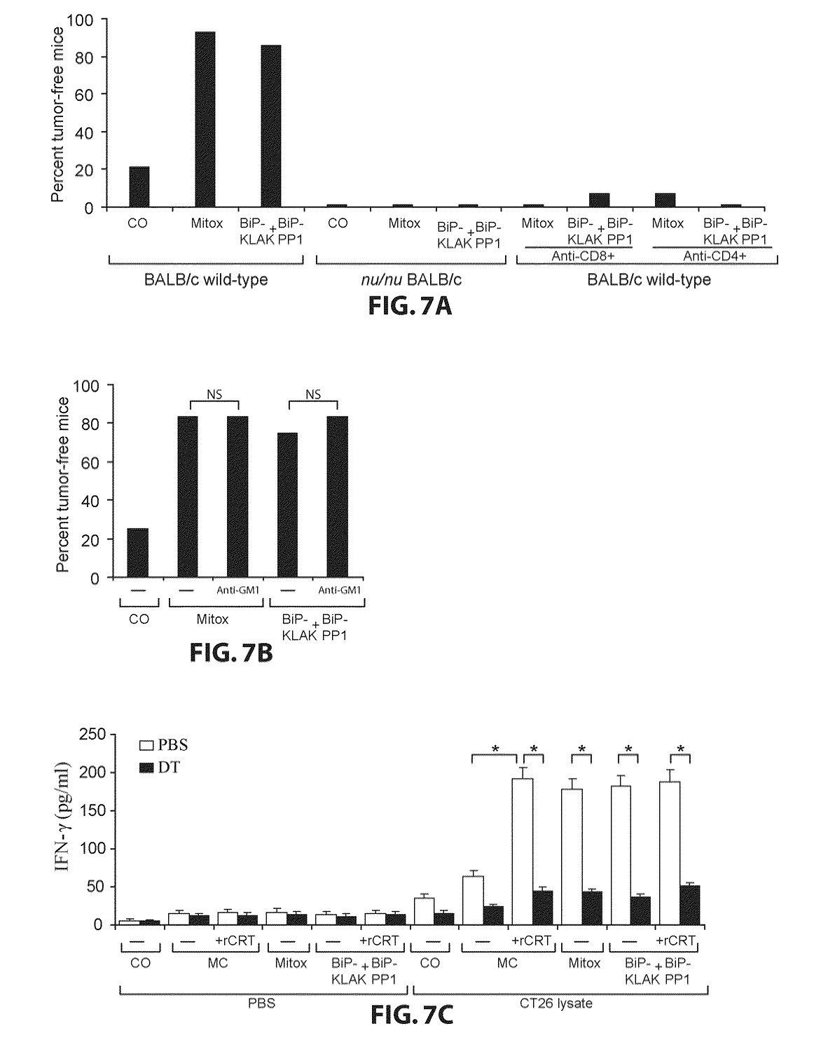

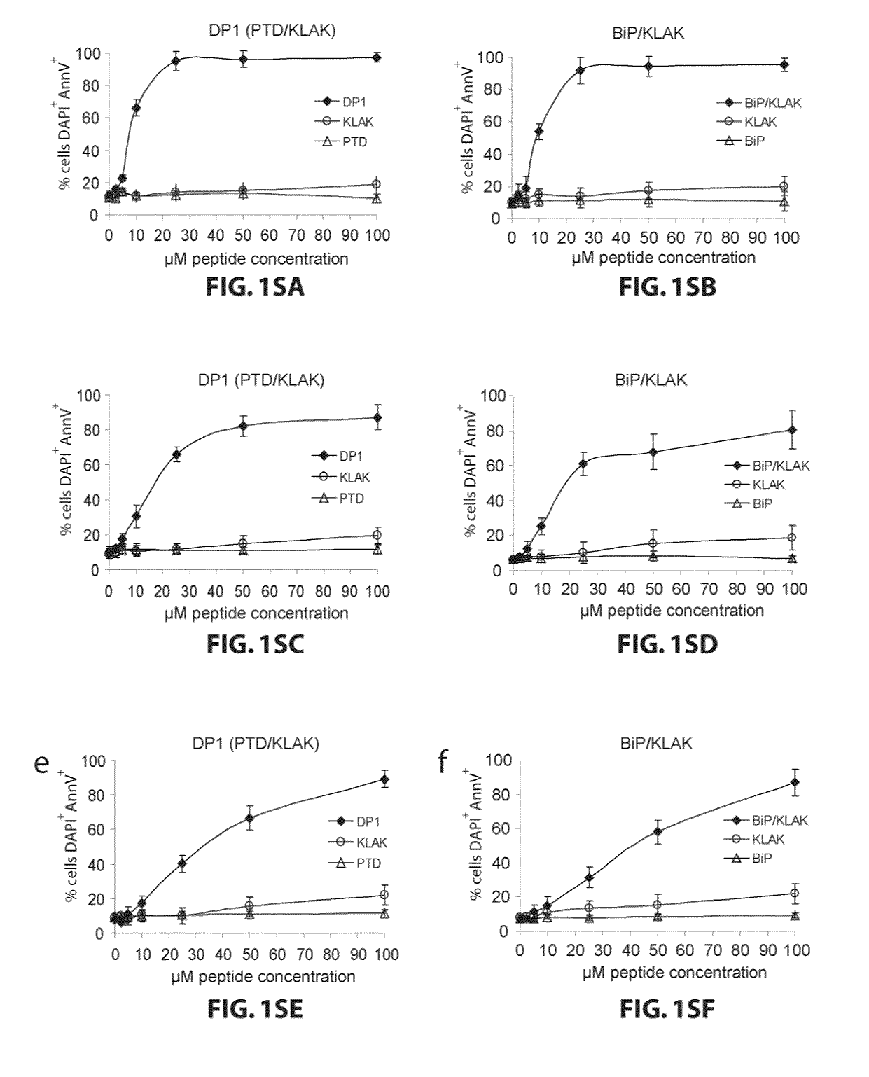

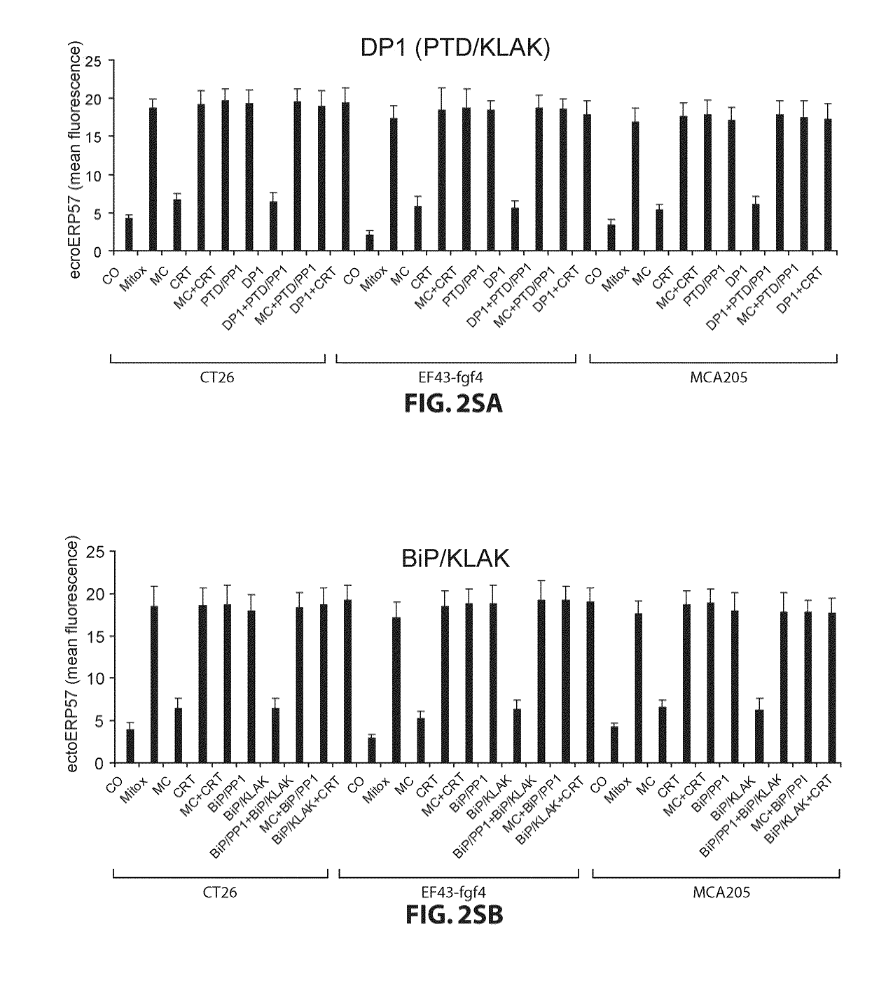

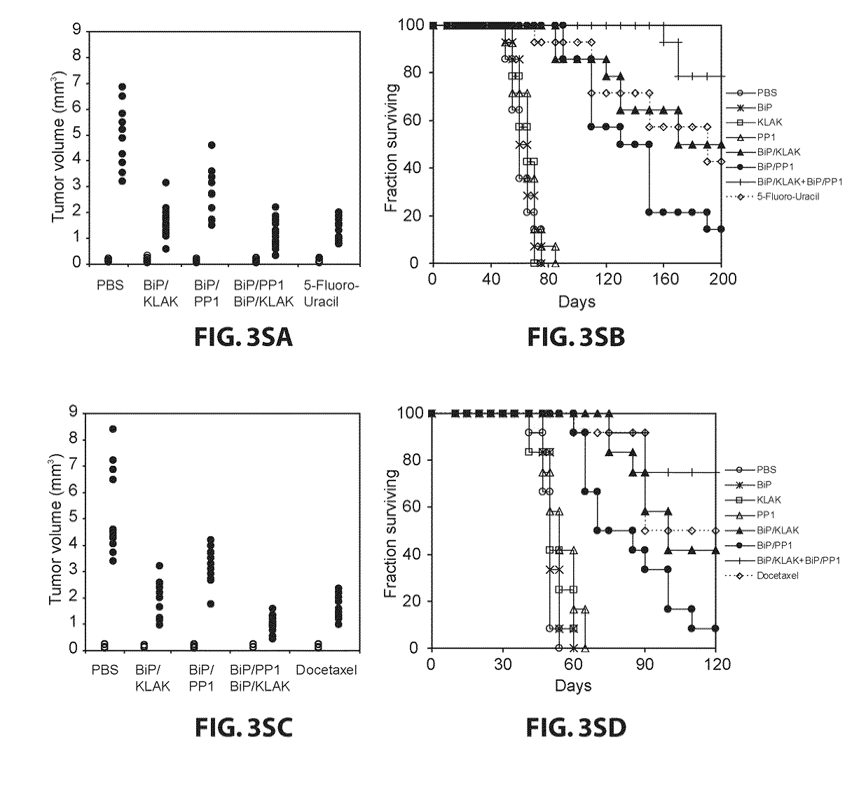

Because I reported recently (a) that ectocalreticulin and ectoERP57 are translocated together to the cell surface in the same molecular complex (1), (b) that the immunogenicity of tumor cells treated with anthracycline, UVC, or irradiation is elicited after their phagocytosis by dendritic cells wherein ectocalreticulin possess a critical function by playing the role of an “eat me signals” to dendritic cells, and (c) that ectocalreticulin dictates the immunogenicity of cancer cell death induced by ectoERP57-ininducer peptides (FIG. 5), I suggest that ectocalreticulin dictates the immunogenicity of ectoERP57-inducer peptides by mediating dendritic cell phagocytosis of treated cells. To evaluate this hypothesis, the level of ectocalreticulin in tumor cells treated by ectoERP57-inducer peptides was measured and correlated to their phagocytosis by dendritic cells. A high level of ectocalreticulin has been found in cells treated with ectoERP57-inducer peptide BiP/PP1 (e.g., BiP/KLAK+BiP/PP1, mitomycin C+BiP/PP1; FIG. 6A). This level was similar to the one observed with immunogenic treatment such as mitoxantrone or to the on observed after the supply of recombinant calreticulin such as mitomycin C+recombinant calreticulin or BiP/KLAK+recombinant calreticulin (FIG. 6A). In conclusion, a strong correlation has been obtained between ectocalreticulin quantity observed in cells treated with ectoERP57-inducer peptides and their phagocytosis by dendritic cells (FIG. 6A). Consequently, I aimed to determine the exact role of ectocalreticulin in the phagocytosis. First, I proceeded to block ectocalreticulin with specific neutralizing antibody of avian origin for calreticulin (refs. 1, 2; antibody cannot interact with mouse Fc receptors). The blocking of ectocalreticulin on mitoxantrone or BiP/KLAK+BiP/PP1-treated cancer cells inhibited their phagocytosis (FIG. 6B). In addition, the use of siRNA calreticulin that abolished ectocalreticulin and the phagocytosis of anthracyclines by dendritic cells (1, 2) inhibits the phagocytosis of BiP/PP1+BiP/KLAK or mitoxantrone-treated cells (FIG. 6C), whereas this inhibition was identically restored for BiP/PP1+BiP/KLAK and anthracyclines-treated cells after the supply of recombinant calreticulin (FIG. 6C). Thus, ectocalreticulin elicits the phagocytosis by dendritic cells of tumor cells treated by ectoERP57-inducer peptides.

### EXAMPLE 7

Crucial Role of Dendritic Cells and T Cells in the Immune Response Elicited by Tumor Cells Treated with EctoERP57-Inducer Peptides.

To understand better how the immunogenic antitumor reaction elicited by ectoERP57-inducers peptides is mediated, the contribution of the different immune system effectors has been evaluated. First, I have checked the impact of T cells (CD4⁺ or CD8⁺) on this immunogenicity. The antitumor vaccination obtained by the treatment with mitoxantrone or with BiP/PP1+BiP/KLAK was completely abrogated in immunodeficient nu/nu mice (FIG. 7A). Similarly, the pharmacologic depletion of T CD4⁺ or T CD8⁺ by in vivo injection of specific neutralizing antibodies but not that of NK cells abolished the antitumor immune reaction elicited by mitoxantrone or BiP/PP1+BiP/KLAK (FIGS. 7A and B). Thus, T cells and not NK cells are required for the antitumor response elicited by cells tread with mitoxantrone or with BiP/PP1+BiP/KLAK. To determine the impact of CD11c⁺ dendritic cells on ectocalreticulin-dependant immune response in vivo, a diphtheria toxin-based inducible system that allows the short-term ablation of dendritic cells in vivo has been used, wherein diphtheria toxin was injected into transgenic mice expressing the diphtheria toxin receptor, especially in dendritic cell (under the control of the CD11c promoter). Lymph node cells from control mice injected with PBS produced interferon-largely after the injection with CT26 cells treated with mitomycin C plus recombinant calreticulin, mitoxantrone, or BiP/PP1+BiP/KLAK followed by in vitro rechallenge with CT26 lysates. Otherwise, lymph node cells from dendritic cell-depleted mice injected with diphtheria toxin did not produce interferon-in response to the same stimuli (FIG. 7C). These data confirm that dendritic cells and T cells and not NK cells play a key role in ectoERP57-inducer peptides-elicited immune response.

What is claimed is:

1. A compound for treating cancer in a mammal, comprising:
an inhibitor that causes an inhibition of an interaction between PP1 and GADD34 of a PP1/GADD34 complex;
The interaction inhibition between PP1 and GADD34 induces, at a first stage; a translocation of ERP57 protein from inside of the cell to the plasma membrane of said at least some of the apoptotic cancer cells;
The increasing in the quantity of cell surface ERP57 protein transforms the non-immunogenic apoptosis of cancer cells in immunogenic apoptosis.
The translocated ERP57 protein is perceptible as “eat me signal” by Dendritic cells of an immune system of the mammal; and wherein the immune system responds to said “eat me signal” by causing the Dendritic cells to phagocyte said at least some of the cancer cells expressed with the translocated ERP57 proteins, thus triggering a specific anti-cancer immune response leading to the eradication of the cancer cells.

2. The inhibitor of claim 1, comprising an inhibitor peptide of the complex PPI/GADD34.

3. The inhibitor peptide of claim 2 includes a complex PP1/GADD34 interaction inhibitor that blocks an interaction between PP1 and GADD34.

4. The peptide inhibitor of claim 3 includes a sequence of amino acids that block the interaction between PP1 and GADD34.
